(19) United States
(12) Patent Application Publication
(10) Pub. No.: US 2005/0182448 A1
(43) Pub. Date: Aug. 18, 2005

(54) Multiple electrode for an oscillation generator and/or oscillation detector

(76) Inventors: Gerd Ohmayer, Haslach (DE); Juergen Dietmeier, Hausach (DE)

Correspondence Address: NATH & ASSOCIATES, PLLC;Sixth Floor, 1030 15th Street, N.W., Washington, DC, 20005, US

(21) Appl. No.: 11/053,661

(22) Filed: Feb. 9, 2005

(30) Foreign Application Priority Data
Feb. 18, 2004 (DE) 10 2004 007 767.3

(51) Int. Cl. A61N 1/00

(52) U.S. Cl. 607/2

(57) ABSTRACT

The invention relates to a multiple electrode (24) for an oscillation generator and/oscillation detector (1) with at least two electrodes (5, 6; 5*, 6*) and a connecting conductor (14; 25, 26) that joins them, such that the two electrodes (5, 6; 5*, 6*) are positioned parallel to each other across a separating distance (h).

The multiple electrode is advantageously designed in that the two electrodes (5, 6; 5*, 6*) and the connecting conductor (14; 25, 26) are formed from a single metal segment.

The invention relates to a multiple electrode for an oscillation generator and/or oscillation detector with the features indicated in the preamble of patent claim 1; to an oscillation generator and/or oscillation detecting device with said multiple electrode; and to a process for manufacturing this multiple electrode.

WO 01/84642 A1 discloses a multilayer piezoceramic component for measuring devices, and a process for manufacturing said device. The multilayer component consists of a plurality of piezoceramic elements positioned in parallel fashion as disk-like oscillating elements stacked one on top of the other. A contact electrode is integrated into the surface of each of these oscillating elements. In each case, two electrodes positioned parallel to each other and belonging to a first, third, and fifth oscillating element are joined together by independent connecting conductors. The electrodes of the second and fourth oscillating elements lying between these are likewise joined together by a connecting conductor. Thus an arrangement is known in which the oscillating elements each exhibit a flat, uppermost electrode layer, such that the electrode layers consisting of a plurality of homopolar electrodes are joined by separate connecting conductors. For the purpose of connection, conductor connectors bent to form a U and acting as separate components are inserted into the stack of oscillating elements. Individual connecting conductors of this kind, to which the identical voltage is applied, are joined together by soldering and through use, e.g., of a printed board assembly.

Such an arrangement of stacked oscillating elements is disadvantageous due to the large number of individual components. In addition to the oscillating elements with the electrode layers on their faces, it is necessary to provide separate connecting conductors. Inserting the connecting conductors into the stack of oscillating elements requires that grooves be formed in the electrode layers. In addition, the connection of more than two electrodes with the same applied voltage requires additional connecting conductors, which must be joined to each other. Furthermore, this kind of multilayer component, with electrodes and connecting conductors soldered together, or with connecting conductors soldered together, can only be employed at lower temperatures, since the solder will otherwise liquefy.

The goal of the invention is to improve a multiple electrode for an oscillation generator and/or oscillation detector with respect to the number of individual elements needed and with respect to the simplicity of the production process.

This goal is achieved with a multiple electrode for an oscillation generator and/or oscillation detector exhibiting the features of patent claim 1 and with a process for producing said multiple electrode exhibiting the features of patent claim 14.

The especially preferred embodiment thus relates to a multiple electrode for an oscillation generator and/or oscillation detector exhibiting at least two electrodes and a connecting conductor that joins them, such that the two electrodes are positioned parallel to each other over a separating distance, and such that the two electrodes and the connecting conductor are formed from a single metal segment. A multiple electrode with this simple design provides a number of advantages. Instead of producing a desired number of individual electrodes, or forming them directly on the oscillating elements, and instead of additionally preparing a plurality of connecting elements, only a single component has to be produced and manipulated. At the same time, this permits the simple connection to a power, or a cable, by means of pressing, shrinking, or welding. In addition to the environmental friendliness afforded by the elimination of solder, it is possible to use the configuration at very high temperatures, at which solder would liquefy. In principle, any desired number of electrodes, with a connecting conductor positioned between each pair, can be formed into a single piece. The formation of flat electrodes also makes it possible to provide the oscillating elements, which will ideally take the form of piezoelements, with contacts that have a large surface area.

The preferred process for manufacturing this kind of multiple electrode comprises the stages of first cutting an electrode structure from a flat piece of metal, such that a connecting conductor is formed between two electrodes, and of then bending the connecting conductor so that the electrodes are positioned in parallel fashion at a predetermined distance one from the other. This process makes it possible to simply bend a multiple electrode into shape from a flat metal element, e.g., one that is stamped out, and that exhibits a plurality of electrodes and connecting conductors that are joined in a single piece; at the same time, the process makes it possible to join together the individual electrodes belonging to the multiple electrode without having to produce and attach separate connecting conductors.

Advantageous elaborations are the subject matter of dependent claims.

A multiple electrode is preferred in which the metal segment is produced from a flat, arc-shaped metal piece. The overall configuration can thus be produced—specifically stamped, etched, or cut out—in a single manufacturing step, including the interdependent electrodes and connecting conductors.

A multiple electrode is preferred in which the electrodes are positioned along a longitudinal axis and the connecting conductor in the uncoiled state connects the electrodes lateral to the longitudinal axis at a given distance or offset. In a particularly simple way this lateral offset makes it possible to subsequently interlock two such multiple electrodes, and allows the given connecting conductors of the two different multiple electrodes to be positioned next to each other without touching.

In a second embodiment a multiple electrode is preferred in which in the uncoiled state the electrodes are positioned one behind the other on the longitudinal axis and the connecting conductor is positioned parallel to the longitudinal axis and next to the electrodes, such that the electrodes are connected to the connecting conductor by a connecting bridge, to form a single piece. Here a multiple electrode is preferred in which the connecting bridges in the multiple electrode's ultimate structure are positioned at the level of the electrodes joined together and positioned in parallel fashion. A multiple electrode is thus created with individual electrodes forming a single piece with a single connecting conductor, such that in the final structure the configuration forms a stalk or stem, with individual electrodes projecting laterally on one or both sides.

According to a particularly preferred initial embodiment a multiple electrode is preferred in which in uncoiled state the electrodes and the connecting conductor are positioned in succession on a flat plane, with the connecting conductor between the electrodes. Here a multiple electrode is preferred in which the two electrodes in the final structure lie parallel to each other and in which the connecting conductor runs between the two and forms an arc. Particularly advantageous here is multiple electrode with a third electrode, which is connected by a second connecting conductor to the second electrode, such that in the ultimate structure the second connecting conductor is positioned on the side opposite the first connecting conductor, relative to the longitudinal axis of the final structure. Thus, in the final structure a multiple electrode according to this embodiment runs in serpentine fashion around and along a central longitudinal axis. It is advantageous that with the bending process the distance separating the individual electrodes positioned adjacent to each other can be readjusted, from a minimal separating distance up to a distance corresponding to the length of the individual connecting conductors running the individual electrodes.

A multiple electrode is preferred in which the electrodes each exhibit a central hole and these holes in the electrode are positioned along a common longitudinal axis in the final structure.

Particularly preferred is an electrode configuration with two such multiple electrodes, each with at least two electrodes, such that the electrodes are positioned parallel to each other along a longitudinal axis and such that one of the electrodes of one multiple electrode is in each case positioned between two electrodes of the other multiple electrode and the connecting conductors of the two multiple electrodes are positioned on a plane whose longitudinal axis runs along or parallel to the longitudinal axis of the electrode configuration and the planes are rotated around the longitudinal axis by a given angle or are offset from the longitudinal axis by a given amount. Thus in one embodiment variant, the two planes formed by the connecting conductors of the multiple electrodes are rotated relative to each other by a given angle, so that the connecting conductors cannot touch each other.

According to another embodiment the two planes of the connecting conductors are laterally offset one relative to other, particularly in lateral, parallel fashion, so that the connecting conductors of the different multiple electrodes cannot touch each other, but nonetheless project laterally from the entire configuration and provide contacts for the corresponding electrodes.

Particularly preferred is an oscillation generator and/or oscillation detector with two electrodes serving as the multiple electrode, and with at least one counter-electrode between the electrodes, where the two electrodes are joined by a connecting conductor that connects them laterally,

and with oscillating elements between adjacent electrodes and the counter-electrode, such that the two electrodes and the connecting conductor are formed into the multiple electrode from a single-piece metal segment or metal arc.

This oscillation generator and/or oscillation detector is advantageously provided with a central hole running through the electrodes and oscillating elements, and with a connecting element that is guided through the holes. The connecting element will advantageously exhibit a central through-hole. This through-hole permits provides central accessibility through the entire configuration, to a body from which oscillations are recorded or to which generated oscillations are transmitted.

An exemplary embodiment will next be described in greater detail on the basis of the drawing. Shown are:

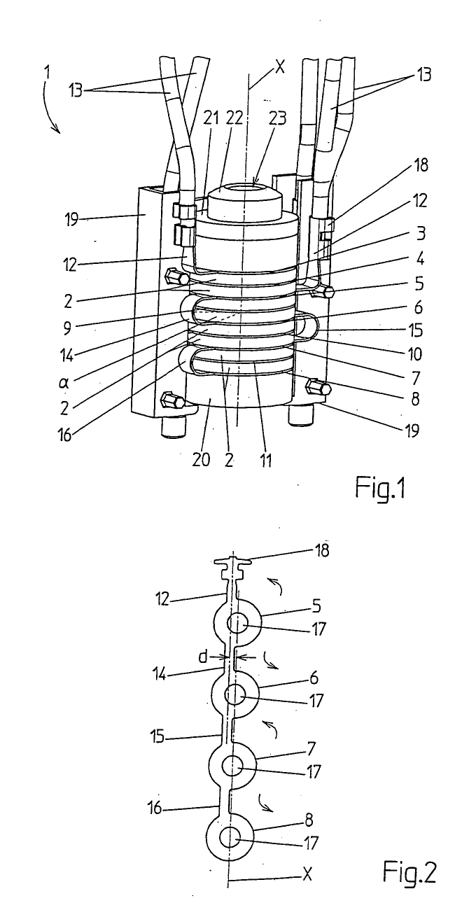

FIG. 1 an oscillation generator and oscillation detector with a configuration of several multiple electrodes

FIG. 2 a multiple electrode, as in FIG. 1, in uncoiled condition

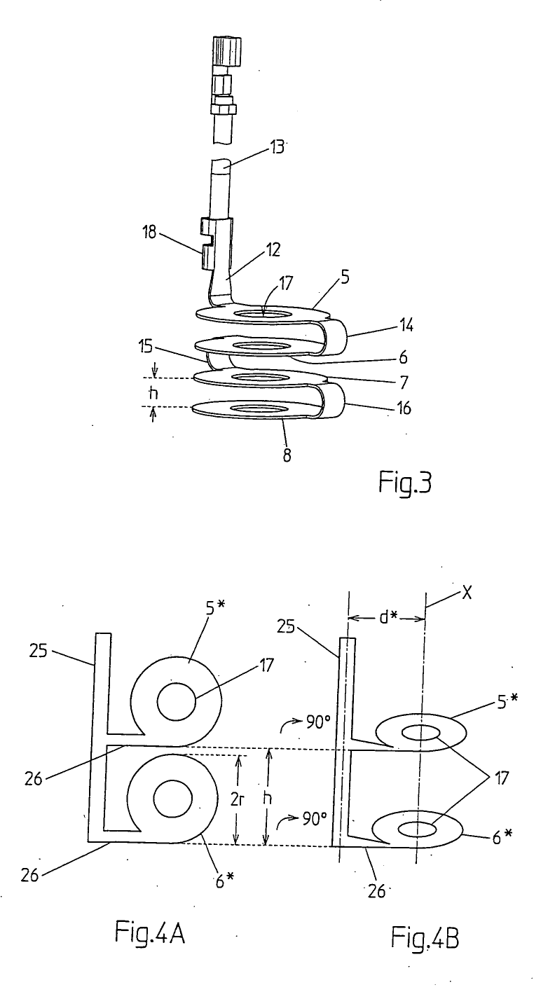

FIG. 3 the multiple electrode of FIG. 3, in its ultimate state

FIG. 4A, a multiple electrode in its coiled state and in its ultimate state, 4B in another embodiment

FIG. 1 shows an oscillation generator and oscillation detector 1 with a plurality of oscillating elements, between which are inserted electrodes and counter-electrodes. This kind of device can be operated as an oscillation generator, as an oscillation detector, or as a combined oscillation generator and detector. In particular, the number of individual oscillating elements 2, ideally peizoceramic oscillating elements, as well as their operation, is possible in other and different configurations. The overall configuration depicted extends cylindrically in the longitudinal direction around a longitudinal axis X. In principle, other cross-sectional designs can be realized.

In the particularly preferred embodiment the oscillation generator and oscillation detector 1 exhibits a plurality of oscillating elements 2. The oscillating elements 2, ideally piezoceramic elements, are disk-shaped, with their faces positioned one above the other, and run in circular fashion around the longitudinal axis X. Positioned between every two oscillating elements 2, as well as on the outside of the stack created by the oscillating elements 2, is an electrode 3, 5-8 or a counter-electrode 4, 9-11. The first block of oscillating elements 2 between the first electrodes 3, 5 and the first counter-electrode 4 forms, e.g., an oscillation detecting unit for detecting oscillations, which are transmitted to the overall device. The second structural group consisting of the other oscillating elements 2 and electrodes 5-8 or counter-electrodes 9-11 form, e.g., an oscillating generator unit for generating oscillations, which are transmitted to an undepicted unit that is positioned on the face. This second group thus consists of a plurality of oscillating elements 2, of which every second oscillating element 2 is to be to the same voltage on a side exhibiting an electrode 5-8. The plurality of these individual electrodes 5-8 thus forms a multiple electrode due to the connecting conductors 14-16 positioned between them. The other side of this group of oscillating elements 2 is wired in corresponding fashion with counter-electrodes 9-11, which also have a common voltage and are connected by connecting conductors. In the depicted exemplary embodiment the connecting conductors of the counter-electrodes 9-11 run inside of two guiding and fastening elements 19 positioned to the side of the overall configuration and consequently are not depicted. The second multiple electrode, which is formed by the counter-electrodes 9-11 and their connecting conductors, in essence corresponds to the design of the first multiple electrode 24, which is formed by the electrodes 5-8 and their connecting conductors 14-16.

The first electrode 5, 9 of such a group of multiple electrodes, or of individual electrodes 3, 4, exhibits an attachment conductor 12, to which a cable 13 for the feed and removal of voltage and current is attached via a clamping section 18.

The overall arrangement of oscillating elements 2, electrodes 3, 5-8, and counter-electrodes 4, 9-11 will ideally be held together by a clamping device. In the exemplary embodiment shown, the clamping device consists of an intitial clamping and oscillation-transmitting element 20 and, beyond the oscillating elements, an intermediate piece and a second clamping element 21, which are clamped by means of, e.g., a tightening bolt 22 or a tightening screw. The depicted tightening bolt 22 runs through a central hole 17, which extends through the entire arrangement consisting of clamping and oscillation-transmitting element, electrodes 4, 9-11, oscillating elements 2, intermediate element, and clamping element 21. This arrangement advantageously provides centralized clamping along the common longitudinal axis X, which is the main oscillating axis. In the depicted exemplary embodiment the tightening bolt 22 running through the center of the configuration exhibits a through-hole or access opening, so that central access is provided to the components and devices on which the oscillation generator and oscillation detector 1 are mounted. As an alternative, other known arrangements for fastening the individual oscillating elements 2 and electrodes and counter-electrodes 3-11 can be employed.

FIGS. 2 and 3 show an especially preferred embodiment of the first multiple electrode 24. FIG. 2 shows the first multiple electrode 24 in the initial step in a process for manufacturing the multiple electrode. The first multiple electrode 24 consists of a flat, single-piece structural element, which is produced from a flat metal arc, or from an electrically conductive material. Production may be performed by, e.g., stamping, etching, or cutting out. The multiple electrode 24 consists of a sequence of electrodes 5-8. Every two adjacent electrodes 5-6, 6-7, 7-8 are connected in single-piece fashion by the connecting conductors 14, 15, or 16 between the electrodes. The first electrode 5 also exhibits an attachment conductor 12, which in the depicted embodiment is provided with brackets to form a clamping section.

According to a particularly preferred embodiment, individual connecting conductors 14-16 and the attachment conductor 12 lie parallel to a central longitudinal axis X and lateral to the longitudinal axis X by an offset distance d, and are positioned between the electrodes 5-8. If the second multiple electrode is designed to have connecting conductors which are offset relative to the opposite side of the longitudinal axis X, then the two multiple electrodes, with their individual electrodes and counter-electrodes, can be positioned between each other, such that the individual connecting conductors of the two multiple electrodes run beside each other and project laterally from the stack of oscillating elements 2 and electrodes 5-8 or counter-electrodes 9-11, without interfering with each other.

FIG. 3 shows a second step in the process for manufacturing this kind of multiple electrode 24. Proceeding from one end of the multiple electrode 24, the individual connecting conductors 14-16 are bent in such a way that the two adjacent electrodes 5-6, 6-7, 7-8 are positioned parallel to each other, with a distance h separating the electrodes of identical polarity. Along with the option of providing the connecting conductors 14-16 with angular bends, uniformly bent connecting conductors 14-16 are especially preferred.

Two multiple electrodes, as a configuration of electrodes 5-8 and counter-electrodes 9-11 positioned between them, are inserted one inside the other in such away that one counter-electrode 9, 10, or 11 is positioned between every two electrodes 5-6, 6-7, 7-8. The connecting conductors 14-16 and the attachment conductor 12 of the first multiple electrode 24 thus formed are so oriented that they run in an initial plane whose longitudinal axis forms a common line of intersection with the longitudinal axis X of the electrode configuration, or runs parallel to that axis X. The second multiple electrode, with the counter-electrodes and the connecting conductors joining them, is oriented accordingly; however, the planes of the two multiple electrodes, with respect to their connecting conductors, are rotated around the longitudinal axis X, one relative to the other, by an angle of α, or are laterally offset from the axis X. In the embodiment shown in FIG. 1 the connecting conductors 14-16 of the first multiple electrode 24 run on a plane that is laterally offset relative to the longitudinal axis X, in front of the depicted guide and fastening elements 19. The connecting conductors of the second multiple electrode run in undepicted fashion on a plane through the longitudinal axis X or parallel to, but offset from, the longitudinal axis X, in or behind the guide and fastening elements 19.

In order to make possible the clamping mechanism according to FIG. 1, the individual electrodes 5-8, as shown in FIGS. 2 and 3, each exhibit a central hole 17 for guiding fastening elements and the like.

According to a second embodiment a plurality of electrodes 5*, 6* is again positioned on a connecting conductor 25, to form a single piece. However, the connecting conductor 25 runs parallel to the individual electrodes 5*, 6*, such that the individual electrodes 5*, 6* are each joined to the single connecting conductor 25 by a connecting segment or connecting bridge 26. The distance between the individual connecting bridges 26 corresponds to the separating distance h between the electrodes in the final structure, after the electrodes 5*, 6* are each bent 90° (FIG. 4B). This embodiment provides an advantage in that despite the single-piece design of the individual electrodes 5*, 6* and the connecting conductors 25, 26, the electrodes 5*, 6* can be easily brought into the desired terminal position; when the multiple electrodes are fitted into each other, the two corresponding connecting connectors can be positioned next to each other or facing each other, as desired—without the need for angular or offsetting adjustment. However, in the simplest form of the second embodiment, the separating distances h of the electrodes 5*, 6* are dependent on the width or radius of the individual electrodes 5*, 6*. For a bilateral arrangement of the electrodes on the connecting conductor this distance is reduced by half.

1. Multiple electrode (24) for an oscillation generator and/or oscillation detector exhibiting at least two electrodes (5, 6; 5*, 6*) and a connecting conductor (14; 25, 26) that joins them, such that the two electrodes (5, 6; 5*, 6*) are positioned parallel to each other over a separating distance (h),
wherein
the two electrodes (5, 6; 5*, 6*) and the connecting conductor (14, 25, 26) are formed from a single metal segment.

2. Multiple electrode according to claim 1, where the metal segment is produced from a flat, arc-shaped metal piece.

3. Multiple electrode according to claim 1, where the electrodes (5, 6; 5*, 6*) are positioned along a longitudinal axis (X) and the connecting conductor (14; 25) in uncoiled condition joins together the electrodes (5, 6; 5*, 6*) to the side of the longitudinal axis (X) at an offset distance (d; d*).

4. Multiple electrode according to claim 1, where in uncoiled state the electrodes (5*, 6*) are arranged one behind the other on the longitudinal axis (X) and the connecting conductor (25) is laterally positioned next to the electrodes (5*, 6*) and parallel to the longitudinal axis (X), and where the electrodes are joined to the connecting conductor (25) by a connecting bridge (26) and form a single piece.

5. Multiple electrode according to claim 4, where in the final structure of the multiple electrode the connecting bridges (26) are positioned on the plane of the interconnected electrodes (5*, 6*), which are positioned parallel one to the other.

6. Multiple electrode according to claim 1, where in uncoiled condition the electrodes (5, 6) and the connecting conductor (14) are positioned one behind the other on a flat plane, with the connecting conductor (14) between the electrodes (5, 6).

7. Multiple electrode according to claim 6, where in the ultimate structure the two electrodes (5, 6) are positioned parallel to each other and the connecting conductor (14) runs between the two in an arc.

8. Multiple electrode according to claim 6, with a third electrode (7), which is joined to the second electrode (6) by a second connecting conductor (15), such that in the final structure the second connecting conductor (15) is positioned on the side opposite the first connecting conductor, relative to the longitudinal axis (X).

9. Multiple electrode according to claim 1, in which the electrodes (5, 6; 5*, 6*) each exhibit a central hole (17) and in the final structure these holes (17) in the electrodes are positioned along a common longitudinal axis (X).

10. Electrode configuration with two multiple electrodes according to claim 1, comprising at least two electrodes (5-8 and 9-11), such that the electrodes are positioned parallel to each other along a longitudinal axis (X), and such that any one of the electrodes (9-11) of one of the multiple electrodes is always positioned between two of the electrodes (5-8) of the other multiple electrode (24), and where the connecting conductors (14-16) of the two multiple electrodes are positioned on a plane whose longitudinal axis runs along or parallel to the longitudinal axis (X) of the electrode configuration, and the planes, one relative to the other, are rotated around the longitudinal axis by an angle (a), or are offset by a distance (d) relative to the longitudinal axis.

11. Level gauge (1) with
two electrodes (5, 6) comprising a multiple electrode (24), such that the two electrodes (5, 6) are joined together from the side by a connecting conductor (14),
at least one counter-electrode (9) between the electrodes (5, 6), and
oscillating elements (2) between any adjacent electrodes (5, 6) and the one or more counter-electrodes (9),
wherein
the two electrodes (5, 6) and the connecting conductor (14) are designed as a multiple electrode in accordance to one of the preceding claims and consist of a single-piece metal segment.

12. Level gauge according to claim 11, with a central hole (17) through the electrodes (5, 6, 9) and the oscillating elements (2), and with a connecting element (22) guided through the holes (17).

13. Level gauge according to claim 12, where the connecting element (22) exhibits a central through-hole (23).

14. A process for manufacturing a multiple electrode according to a claim 1, with the following steps:
separating an electrode structure from a flat metal piece, such that a connecting conductor (14-16) is formed between two electrodes (5-6, 6-7, 7-8), and
bending the connecting conductor (14-16) in such a way that the electrodes (5-8) are positioned parallel to each other and are separated by a a given distance (h).
